(19) United States
(12) Patent Application Publication
(10) Pub. No.: US 2009/0311141 A1
(43) Pub. Date: Dec. 17, 2009

(54) IMMUNOCHROMATOGRAPHIC STRIP FOR USE IN IMMUNOASSAY AND KIT COMPRISING THE SAME

(76) Inventor: Jae-Ku Park, Gyeonggi-do (KR)

Correspondence Address: BERLINER & ASSOCIATES, 555 WEST FIFTH STREET, 31ST FLOOR, LOS ANGELES, CA, 90013, US

(21) Appl. No.: 12/374,590

(22) PCT Filed: Aug. 2, 2007
(86) PCT No.: PCT/KR2007/003722

§ 371 Date: Jul. 23, 2009

(30) Foreign Application Priority Data
Aug. 3, 2006 (KR) 10-2006-0073460

(51) Int. Cl. G01N 33/53

(52) U.S. Cl. 422/56

(57) ABSTRACT

The present invention relates to an immunochromatographic strip and a kit comprising the same. The immunochromatographic strip according to the present invention is characterized in that it comprises a) an adhesive plastic backing, b) a sample pad receiving a liquid sample to be analyzed, which is attached on the surface of the top portion of the adhesive plastic backing, a conjugate pad coupled to the sample pad, which contains a conjugate specifically binding to an analyte in the liquid sample from the sample pad, d) a signal detection pad coupled to the conjugate pad, which comprises a signal detection zone to detect the presence of the analyte in the liquid sample and a control zone to confirm whether the liquid sample chromatographically migrates regardless of the presence of the analyte, and e) an absorbent pad positioned downstream of the signal detection pad, which absorbs the liquid sample after the completion of the signal detection reaction and comprises a porous backing and an absorbent dispersed, adsorbed, or coated into the pores of the porous backing. It is more preferable that the absorbent pad further comprises a film layer attached on the top portion of the porous backing. It is most preferable that the film layer attached on the top portion of the porous backing is a porous membrane. The absorbent pad having the above-described structure efficiently absorbs the liquid sample after the completion of the signal detection reaction. As a result, the chromatographic flow is improved, test errors caused by the backflow of liquid sample are reduced, background clearance is improved, and the result of signal detection is consistently maintained. More significantly, with the help of the absorption ability improved by the absorbent which is dispersed into the porous backing, more preferably adsorbed, or coated onto fibers of the porous backing, the immunochromatographic strip and kit comprising the same absorb moisture in air quickly and sufficiently. Thus, in addition to improved performance, there is no need to package the strip or the kit with an additional absorbent bag in a pouch, which makes the packaging process simple, thereby improving the speed of automatic packaging process.

### TECHNICAL FIELD

The present invention relates to an immunochromatographic strip and a kit comprising the same. More specifically, the present invention relates to an immunochromatographic strip used for immunoassay and a kit comprising the same.

### BACKGROUND ART

Immunochromatographic assay is a rapid method for qualitative and quantitative detection of analytes using the property that biological or chemical materials specifically bind with one another. Typically, as the immunochromatographic assay device, an assay strip or an immunoassay kit, which is configured as the assay strip mounted in a plastic housing, is used.

FIG. 1 is a cross-sectional view showing a representative embodiment of the conventional immunochromatographic assay strip. As shown in FIG. 1, the assay strip 10 used in the immunochromatographic assay includes a sample pad 21, a conjugate pad 22, a signal detection pad 23, and an absorbent pad 24 on an adhesive plastic backing 11. The sample pad 21 absorbs a liquid sample (or analyte) and provides a uniform flow of the liquid sample. The conjugate pad 22 contains fluid conjugates specifically binding to analytes in the liquid sample, and while the liquid sample introduced to the sample pad 21 flows along the conjugate pad 22, the analyte and the fluid conjugate specifically bind to each other. The signal detection pad 23 typically comprises a detection zone 23a and a control zone 23b. The detection zone 23a is a zone for confirming the presence of analyte in the liquid sample, and the control zone 23b is a zone for confirming whether the liquid sample normally passes through the detection zone 23a. The absorbent pad 24 is placed on the top portion of the signal detection pad 23. The absorbent pad 24 absorbs the liquid sample having passed through the signal detection pad 23, and facilitates capillary flow of the liquid sample in the assay strip 10. That is, the sample pad 21, the conjugate pad 22, the signal detection pad 23, and the absorbent pad 24 are attached to the adhesive plastic backing 11 in this order, so as to form the assay strip 10. The immunoassay is performed using the assay strip 10, in which the liquid sample flows from the sample pad 21 to the absorbent pad 24 via the signal detection pad 23, and the signal is detected in the signal detection pad 23. In other modified method, the conjugate and the signal detection material can be integrated in a single porous pad. Further, the sample pad, the conjugate pad, the signal detection pad, and the absorbent pad can be overlapped to be arranged or consecutively arranged by a predetermined spacing on a plastic backing. In the latter case, the liquid sample flows by a capillary action by virtue of other mediator to the sample pad, the conjugate pad, and the signal detection pad.

After the signal detection is completed in the signal detection pad 23, the remaining liquid is absorbed in the absorbent pad 24. At this time, if the absorbent pad 24 does not have a sufficient absorption ability, the liquid sample or conjugate adsorbed in the absorbent pad 24 flows backward to the signal detection pad 23, which is further forced by dryness of the signal detection pad 23 with the lapse of time. The liquid sample or conjugate adsorbed in the absorbent pad 24 flows backward to the signal detection pad 23, and attached to the control zone 23b or detection zone 23a of the signal detection pad 23, so as to cause a problem that the test result can not be maintained consistently, and sometimes lead to a false test result by the nonspecific reaction with the signal detection material of the detection zone 23a. Accordingly, there is a high correlation between the absorption ability of the absorbent pad 24 and background clearance.

Meanwhile, the assay strip 10 is sensitive to moisture. In particular, a false test result can be caused when the signal detection materials present in the conjugate pad and the detection zone 23a are inactivated by moisture, which is a critical drawback to the assay strip. Therefore, the assembly of the assay strip is carried out in a space with a relative humidity of 20% or less and, in order to minimize the effect of moisture, the assay strip 10 is generally packed with an absorbent bag. Such packaging method is identically applied to a kit comprising the assay strip 10 installed in a plastic housing.

FIG. 2 is a perspective view showing a specific embodiment of the packaging of the kit comprising the strip used in the immunochromatographic assay. As shown in FIG. 2, the conventional immunochromatographic kit 1′, which is available from the market, is packaged in a pouch 3′ with an absorbent bag 2′. However, the process of packaging with the absorbent bag 2′ reduces the efficiency of packaging.

The absorbent bag 2′ is typically packed with silica gel. However, silica gel absorbs moisture slowly, and its absorption rate is relatively low. Thus, in order to effectively remove the moisture, an improved absorbent having high absorption ability is required.

Specifically, the absorbent comprising silica gel has properties of absorbing 8 to 15% of moisture under a relative humidity of 20% and absorbs moisture very slowly.

Further, there is a problem in that substances harmful to the environment are generated during the production process of silica gel, thereby requiring high wastewater treatment costs.

### DISCLOSURE OF INVENTION

### Technical Problem

It is an object of the present invention to provide an immunochromatographic strip and a kit comprising the same, in which the backflow of a liquid sample or conjugate absorbed in an absorbent pad to a signal detection pad is prevented, so that the result of immunochromatographic assay can be maintained consistently for a long period of time.

It is another object of the present invention to provide an immunochromatographic strip and a kit comprising the same, in which, in addition to the backflow prevention, an absorbent bag comprising silica gel required in the conventional packaging process is not used, and an absorbent capable of rapidly and powerfully absorbing moisture in the air trapped in a packaging material is provided inside the assay strip, so that the kit can be singly packaged in the packaging material.

When the immunochromatographic strip or the kit comprising the same according to the present invention is used, the backflow of a liquid sample or conjugate absorbed in an absorbent pad to a signal detection pad is prevented, so that the false test results due to the backflow of the liquid sample or conjugate can be prevented.

Furthermore, in addition to the backflow prevention, the immunochromatographic strip and the kit comprising the same dose not require an absorbent bag in the packaging process, so that the production process can be simplified to obtain an advantage at the expense. Further, in the immunochromatographic strip or the kit comprising the same according to the present invention, the assay strip or kit can be singly packaged in the packaging material without the absorbent bag, whereby the automatic and quick packaging of the products can be easily realized.

### Technical Solution

The immunochromatographic strip according to the present invention is characterized in that it comprises a) an adhesive plastic backing, b) a sample pad receiving a liquid sample to be analyzed, which is attached on the surface of the top portion of the adhesive plastic backing, c) a conjugate pad coupled to the sample pad, which contains a conjugate specifically binding to an analyte in the liquid sample from the sample pad, d) a signal detection pad coupled to the conjugate pad, which comprises a signal detection zone to detect the presence of the analyte in the liquid sample and a control zone to confirm whether the liquid sample chromatographically migrates regardless of the presence of the analyte, and e) an absorbent pad positioned downstream of the signal detection pad, which absorbs the liquid sample after the completion of the signal detection reaction and comprises a porous backing and an absorbent dispersed, adsorbed, or coated into the pores of the porous backing. In this connection, the sample pad, the conjugate pad, the signal detection pad, and the absorbent pad can be attached with one other by overlapping in order. Alternatively, any one or all of the sample pad, the conjugate pad, the signal detection pad, and the absorbent pad can be arranged with a predetermined spacing on the plastic backing, so that the liquid sample is allowed to move by a capillary action by virtue of other mediator. According to a more preferred embodiment, the absorbent pad further comprises a film layer attached on the top portion of the porous backing. It is most preferable that the film layer attached on the top portion of the porous backing is a porous membrane.

### Advantageous Effects

The immunochromatographic strip and kit comprising the same according to the present invention has functions of absorbing a liquid sample, reduces test errors caused by the backflow of conjugate, because an absorbent powerfully absorbs the liquid sample to prevent the liquid sample from flowing backward to a signal detection pad, consistently maintains the test result even after the signal detection pad is dried, and absorbs moisture in air, so that a process of packaging the strip or kit with an additional absorbent bag in a pouch is not needed, and a simplification of the packaging process can be achieved, thereby improving the speed of automatic packaging process.

In particular, the immunochromatographic strip and kit comprising the same according to the present invention comprise an absorbent pad containing a porous backing and an absorbent dispersed, adsorbed, or coated into the pores of the porous backing. It is more preferable that the absorbent pad further comprises a film layer attached on the top portion of the porous backing. It is most preferable that the film layer attached on the top portion of the porous backing is a porous membrane. The absorbent pad having the above-described structure efficiently absorbs the liquid sample after the completion of the signal detection reaction. As a result, the chromatographic flow is improved, test errors caused by the backflow of liquid sample are reduced, background clearance is improved, and the result of signal detection is consistently maintained. More significantly, with the help of the absorption ability improved by the absorbent which is dispersed, adsorbed, or coated into the porous backing, the immunochromatographic strip and kit comprising the same sufficiently absorb moisture in air. Thus, there is no need to package the strip and kit with an additional absorbent bag in a pouch, which makes the packaging process simple, thereby improving the speed of automatic packaging process. Further, since the kit has a powerful moisture-absorption ability, its performance can be effectively protected from moisture trapped during the production process.

### BRIEF DESCRIPTION OF THE DRAWINGS

FIG. 1 is a cross-sectional view showing a representative embodiment of the conventional immunochromatographic assay strip.

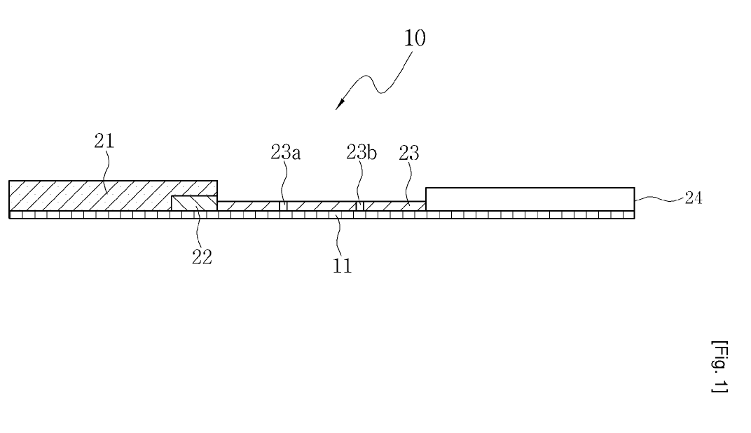

FIG. 2 is a perspective view showing a specific embodiment of the packaging of the kit comprising the strip used in the conventional immunochromatographic assay.

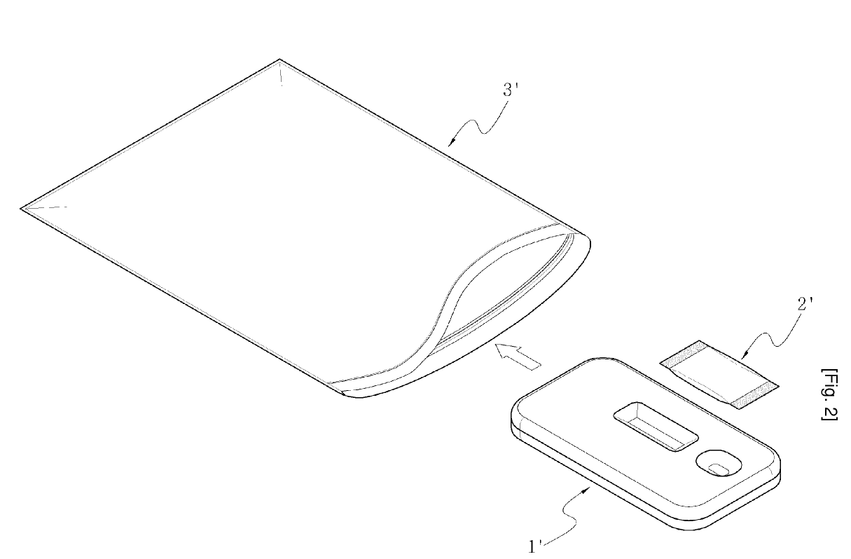

FIG. 3 is a cross-sectional view showing a preferred embodiment of the immunochromatographic assay strip according to the present invention.

FIG. 4 is a perspective view showing a specific embodiment of directly applying a test sample to the immunochromatographic assay strip according to the present invention.

FIG. 5 is a perspective view showing a specific embodiment of applying the immunochromatographic assay strip according to the present invention to a kit.

FIG. 6 is a perspective view showing a specific embodiment of the packaging of the kit comprising the strip used in the immunochromatographic assay according to the present invention.

### MODE FOR THE INVENTION

Hereinafter, the present invention will be described in detail with reference to the accompanying drawings.

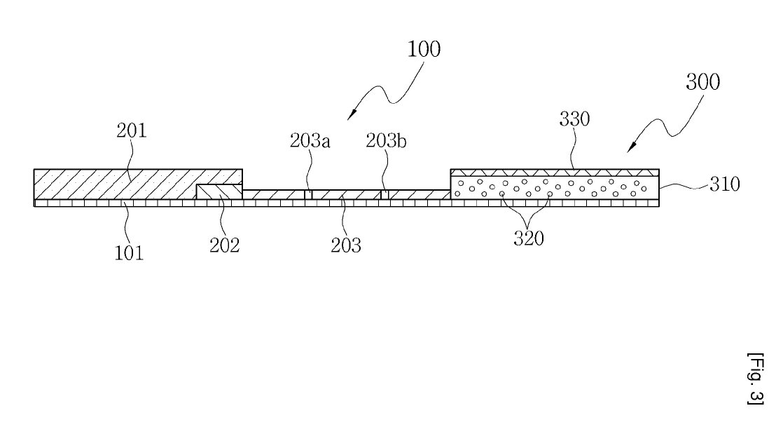

FIG. 3 is a cross-sectional view showing a preferred embodiment of the immunochromatographic assay strip according to the present invention. As shown in FIG. 3, the immunochromatographic assay strip 100 according to the present invention comprises an adhesive plastic backing 101, and a sample pad 201, a conjugate pad 202, a signal detection pad 203 and an absorbent pad 300 attached on the adhesive plastic backing.

First, a liquid sample is supplied to the immunochromatographic strip 100 at the sample pad 201. Such liquid sample as whole blood, plasma, serum, tear, saliva, urine, nasal fluid, and body fluid can be analyzed by the immunochromatographic assay using the immunochromatographic strip 1. The sample pad 201 may further have a function of filtering, in order to improve the selectivity to analytes or minimize the effects caused by interfering substance. For example, in the case of using whole blood as a liquid sample, a denser pad capable of filtering red blood cells may be used or an auxiliary material to facilitate the function of filtering may be further included. If necessary, an auxiliary pad, which contains a material capable of improving the reaction between the analyte and the conjugate or excluding the effects caused by interfering substance, may be further provided upstream of the sample pad 201.

The liquid sample introduced at the sample pad 201 migrates to the conjugate pad 202 positioned upstream of the sample pad 201 by chromatographic flow. The conjugate pad 202 contains conjugates specifically binding to the analyte in the liquid sample. The conjugates can be appropriately selected depending on the type of analyte. The conjugates are labeled with gold particles, latex particles, fluorescent materials, enzymes or the like.

The liquid sample passed through the conjugate pad 202 migrates to the signal detection pad 203. The signal detection pad 203 comprises a signal detection zone 203a detecting the presence of the analyte in the liquid sample and a control zone 203b confirming whether the assay kit is normally operated regardless of the presence of the analyte. For this, the signal detection zone 203a is preferably coated with a material (or signal detection material) selectively and specifically binding to the products resulting from the binding between the analyte and the conjugates in the conjugate pad 202, and the control zone 203b is preferably coated with a material specifically binding to the conjugates in the conjugate pad 202. The signal detection pad 202 consists of a porous membrane pad, and may be formed from nitrocellulose, cellulose, polyethylene, polyethersulfone, nylon or the like.

The most important feature according to the present invention is the absorbent pad 300 positioned upstream of the signal detection pad 203. According to the present invention, the absorbent pad 300 comprises a porous backing 310 and an absorbent 320 dispersed, adsorbed, or coated into the pores of the porous backing 310. Preferably, the absorbent 320 is adsorbed or coated into the porous backing 310. Although FIG. 3 illustrates that the absorbent 320 is present in the form of a fine powder inside the porous backing 310, it is just for better understanding of the present invention. It is most preferable that the absorbent 320 is adsorbed, or partially or completely coated on fibers of the porous backing. In the case where the absorbent 320 is dispersed in the form of the fine powder in the pores of the porous backing 310, there is a high possibility that the fine powder can leak out of the porous backing 310. In a specific example of the present invention, when the silica gel absorbent 320 was dispersed in the form of the fine powder in the pores of the porous backing 310, it was found that the fine silica gel powder leaks out during the production process. The porous backing 310 can be formed from fiber, rubber, or polymer. Examples of the fiber include cellulose, acrylic fiber, polyester fiber, glass fiber, wool fiber, silk fiber, cotton fiber, flax fiber, and pulp. Alternatively, it can be formed from natural rubber, styrene-butadiene rubber, styrene-ethylene-butadiene-styrene copolymer, butyl rubber, ethylene-propylene rubber, ethylene-propylene-diene monomer rubber, ethylene-vinyl acetate copolymer, styrene isoprene block copolymer, styrene butadiene block copolymer, ethylene-acrylate copolymer, butadiene-acrylonitrile copolymer, poly-norbornene, polyisoprene, polyisobutylene, polychloroprene, polybutadiene, polystyrene, polyolefin, polyamide, polyvinylchloride, unsaturated polyester, phenol resin, polyurethane, high density polyethylene, polymer polyethylene, nylon, polyether sulfone, polypropylene or the like. Pulp is preferable. Pulp is more porous than other synthetic fibers or natural fibers, and is very solid as a backing. Therefore, it has excellent ability to hold the absorbent, the moisture absorption ability is more improved by wide dispersion of the absorbent, and the liquid sample can be rapidly and effectively absorbed, which is a basic function of the absorption pad.

The absorbent 320 is dispersed, adsorbed or coated into the pores of the porous backing 310. Examples of the absorbent include diatomite, silicon dioxide (silica gel), aluminum oxide (activated alumina), calcium sulfate, calcium chloride, calcium bromide, lithium chloride, alkaline earth oxide, calcium carbonate, copper sulfate, zinc chloride, zinc bromide, magnesium oxide, magnesium chloride, zeolite, montmorillonite clay, vermiculite, bentonite, lime, dolomite, and gypsum. The absorbent is preferably selected from the group consisting of magnesium chloride, calcium chloride, diatomite, bentonite, lime, dolomite, gypsum, and silica gel, or a mixture thereof in a predetermined ratio. It is most preferable that the absorbent is selected from the group consisting of magnesium chloride, calcium chloride, and a mixture thereof. The absorbent pad 204, in which the absorbent 320 is dispersed, adsorbed, or coated into the pores of the porous backing 310, is prepared in the state of liquid, suspension, or sol by mixing the absorbent with a suitable solvent, and then the porous backing 310 is impregnated therewith, so that the absorbent soaks through evenly, and is dried at 100 to 400° C. (moisture content of 0.5% or less). It is most preferable that the absorbent pad is impregnated with a liquid absorbent solution. A solution was prepared by dissolving magnesium chloride, calcium chloride, and a mixture thereof in water. The porous backing 310 was impregnated with the obtained absorbent solution, and dried at 100 to 400° C. (moisture content of 0.5% or less), resulting in the absorbent pad 204 having excellent performance (see examples below).

According to a more preferred embodiment of the present invention, the absorbent pad 300 comprises a porous backing 310, an absorbent 320 dispersed, adsorbed, or coated into the pores of the backing 310, and a film layer 330 attached on the surface of the top portion of the backing 310. The film layer 330 functions to prevent the leakage of the absorbent 320 adsorbed or coated on the porous backing 310, and to make its handling easy, in the case where the immunoassay strip 100 is used without being contained in a kit. It is most preferable that the film layer 330 is a porous membrane. The porous membrane can be made of polyethylene, polypropylene, nylon, polyvinylidene fluoride, polyether sulfone, polytetrafluoroethylene or the like. The film layer 330 formed from the porous membrane facilitates the rapid release of the liquid sample absorbed in the absorbent pad 300 after the completion of the immunomicroassay, thereby improving background clearance. Specifically, the absorbent pad 300 comprising the absorbent dispersed, adsorbed, or coated into the porous backing 310 prevents the backflow of the liquid sample to the signal detection pad as the absorbent powerfully absorbs the liquid sample. Therefore, background clearance is induced. Accordingly, the test result is easily read, test errors caused by the backflow of conjugate are reduced, and the test result can be consistently maintained, even after the signal detection pad 203 is dried. In addition to the above-described functions, the film layer 330 comprising the porous membrane facilitates the efficient absorption of moisture in air by the absorbent 320 adsorbed or coated into the porous backing 310, thereby effectively preventing damages to the signal detection zone 230a or control zone 230b caused by moisture during the packaging step. Furthermore, the film layer removes the inconvenience of having to package an immunochromatographic strip or a kit comprising the same with an additional absorbent bag during the packaging process.

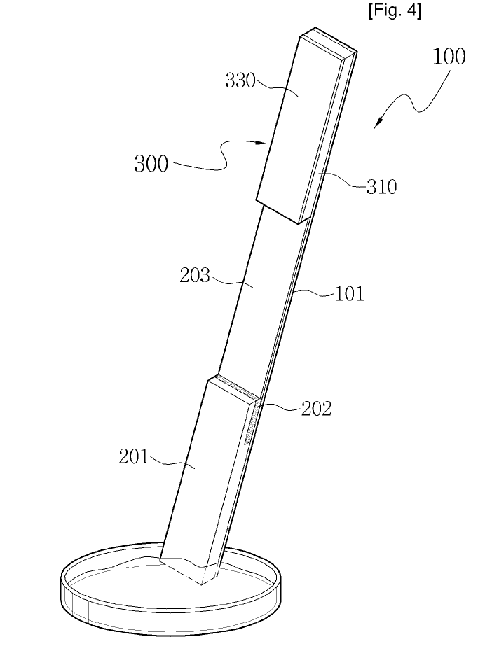

FIG. 4 is a perspective view showing a specific embodiment of directly applying a test sample to the immunochromatographic assay strip according to the present invention. As shown in FIG. 4, while a user grips the adhesive plastic backing 101 and the film layer 303, immunoassay is performed by directly contacting the immunochromatographic strip 100 according to the present invention with a liquid sample to be analyzed.

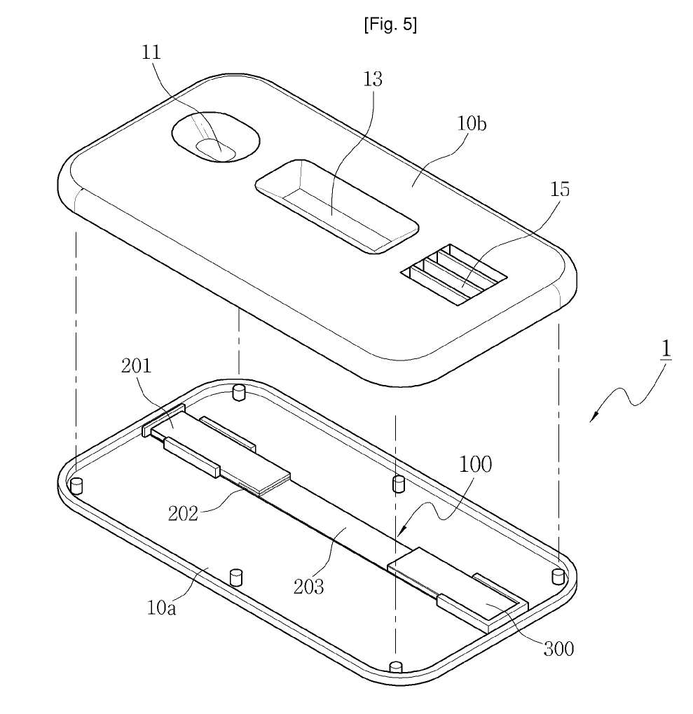

FIG. 5 is a perspective view showing a specific embodiment of applying the immunochromatographic assay strip according to the present invention to a kit. As shown in FIG. 5, the kit used in the immunochromatographic assay comprises a bottom housing 10b to accommodate the immunochromatographic strip 100 and a top housing 10a engaged with the bottom housing. The top housing 10a comprises a first through-hole (or sample through-hole) 11 formed on the top portion of the sample pad 201 in order to receive a liquid sample and a second through-hole 13 formed on the top portion of the signal detection pad 203 in order to observe the result of the immunoassay in the signal detection pad 203. It is more preferable that the top housing 10a further comprises a third through-hole 15 formed on the top portion of the absorbent pad 300. The third through-hole 15 formed on the top portion of the absorbent pad 300 facilitates the absorption of moisture contained in the pouch or the like by the absorbent 320 in the absorbent pad 300, prior to the beginning of immunochromatographic assay using the kit 1. The film layer 330 formed from the porous membrane in the absorbent pad 300 facilitates the moisture absorption. Therefore, there is no need to package the kit with an additional absorbent bag in a pouch. Furthermore, the third through-hole 15 formed on the top portion of the absorbent pad 300 facilitates the evaporation of the liquid sample absorbed in the absorbent pad 300, after starting immunochromatographic assay using the kit 1.

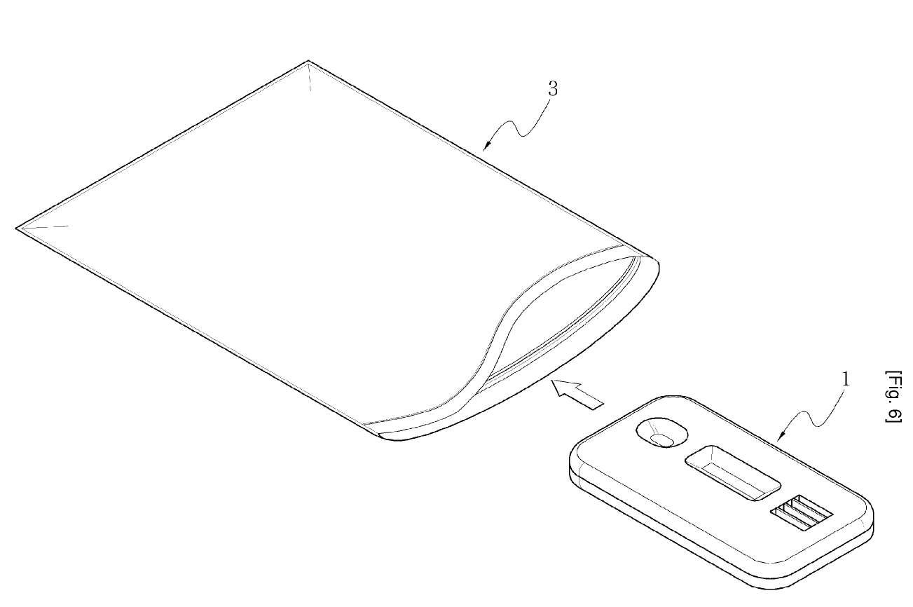

FIG. 6 is a perspective view showing a specific embodiment of the packaging of the kit comprising the strip used in the immunochromatographic assay according to the present invention. As described above, the strip or the kit comprising the same used in the immunochromatographic assay according to the present invention can be packaged in a pouch 3 and can be provided for sale without additional absorbent bag, which makes the packaging process simple, thereby improving the speed of automatic packaging process.

The immunochromatographic strip and the kit comprising the same according to the present invention can be employed for detecting pathogens such as hepatitis C, hepatitis B, influenza virus, avian influenza virus, rotavirus, AIDS, syphilis, chlamydia, malaria, typhoid fever, helicobacter pylori, tuberculosis, SARS, dengue fever, and leprosy and the presence of antibodies using a sample such as whole blood, plasma, serum, tear, saliva, urine, nasal fluid, and body fluid from human or animal. Further, they can be employed in a variety of quick diagnoses including; detecting the presence of pregnancy, ovulation, cancer markers, cardiac markers or the like, detecting whether a subject is under the influence of narcotic drugs such as marihuana, philopon, opium, amphetamines, morphines, and cocaine, confirming bioterrorism by the detection of ricin, anthrax, brucella, botulinum, vaccinia, salmonella, cholera, Staphylococcal enterotoxin B, tularemia or the like, and confirming food-poisoning bacteria such as salmonella, Vibrio campylobacter, enterohemorrhagic Escherichia coli, and Yersinia from foods.

Hereinafter, the present invention will be described in more detail with reference to examples. However, the scope of the present invention is not limited to these examples.

### EXAMPLE 1

5%, 10%, and 20% of magnesium chloride and calcium chloride (weight/volume) were dissolved in water, and paper sheets (0.5×15×2 mm) made of pulp were soaked with each 2 ml of the solutions. The paper sheets were dried at 100° C. for 1 hour, and then a moisture absorption test was performed under a relative humidity of 20%. Further, the magnesium chloride and calcium chloride solutions were mixed with each other in a ratio of 1:1, and then each of the paper sheets (0.5×15×2 mm) made of pulp were soaked with 2 ml of the mixture. The paper sheets were dried at 100° C. for 1 hour, and then a moisture absorption test was performed under a relative humidity of 20%. The highest absorption rate of 15% was found in the solution of 20% concentration among the solutions of calcium chloride, magnesium chloride, and the mixture thereof prepared by mixing with the same amount. The result is much better than the absorption rate of 0.52% of silica gel, which had been left to stand under a relative humidity of 20% for 1 hour, and is comparable to the maximum absorption rate of silica gel (8-15%).

Further, since the absorbent comprising calcium chloride, magnesium chloride, and the mixture thereof is very well absorbed in pulp, fine powders were not observed on the outside after being dried.

TABLE 1

| Material | Concentration (%, W/V) | Absorption rate (%) |
|---|---|---|
| Calcium chloride | 40 | 14.4 |
| | 20 | 14.9 |
| | 10 | 7.6 |
| Magnesium chloride | 40 | 9.9 |
| | 20 | 15.7 |
| | 10 | 10.7 |
| Calcium chloride: | 20:20 | 10.7 |
| magnesium chloride | 10:10 | 14.4 |
| | 5:5 | 8.2 |

Further, the fine powders of diatomite, bentonite, dolomite, gypsum, and silica gel were mixed with the magnesium chloride and calcium chloride solutions, and paper sheets made of pulp were soaked therewith to perform tests. Results similar to the above absorption rate test were obtained. However, the fine powders were not completely dissolved in water, and existed in the form of fine particles. Further, the fine powders were not evenly absorbed in the pulps, and after drying, they were off the pulps.

### EXAMPLE 2

### Test of Canine Parvovirus using Immunochromatographic Strip and Kit

(A) Antibody-Immobilized Nitrocellulose Pad

Monoclonal antibodies against canine parvovirus and goat anti-mouse immunoglobulin antibodies were diluted with a phosphate buffer solution, and were sprayed at the position of the test line and control line of a nitrocellulose pad using a sprayer. Then, the pad was dried in a 37° C. incubator to be immobilized.

(B) Production of Antibody-Gold Conjugate Pad

Monoclonal antibodies against canine parvovirus were added to 1 ml of a colloidal gold aqueous solution, in which pH was adjusted to 8.9, so as to be surface-adsorbed, and then blocked with 1 wt % of bovine serum albumin. The conjugate solution was centrifuged at 12,000 rpm with a centrifuge, and the supernatant was removed. The precipitate was resuspended in a phosphate buffer containing 0.7 wt % of bovine serum albumin and 0.5 wt % of sucrose to until the absorbance became 1, and aliquotted in a glass fiber pad with a ratio of 80 □/25 mm². The pad containing the gold colloid-antibody conjugates was dried in a dehumidifier for 5 hours, and then the dried pad was cut to a size of 0.5×300 mm.

(C) Production of Absorbent Pad Containing Absorbent

Pulp having a thickness of 1 mm was impregnated with an aqueous solution containing 10 wt % calcium chloride, and the pulp was left to be evenly soaked with the absorbent solution. Then, the pulp was dried at 100° C. for 1 hour, and, after attaching a porous membrane (polyethylene) on one side of the pulp, was cut to a size of 20×300 mm.

(D) Production of Immunochromatographic Strip and Kit Comprising the Same

The pads produced in the steps of A, B, and C and a sample pad were attached on a polypropylene plate in order, on which an adhesive had been applied. That is, the sample pad, the antibody-gold conjugate pad, the antibody immobilized nitrocellulose pad, and the absorbent pad obtained above were attached by overlapping in order. The strip was cut to a size of 0.5×70 mm, and packaged in an aluminum pouch. The same strip was mounted in a plastic housing to produce a kit, and then packaged in an aluminum pouch. At this time, an additional absorbent bag was not used. The immunochromatographic strip and the kit comprising the same were stored for 1 month, 6 months, 12 months, 18 months, and 24 months, and then used for the test of each one of positive and negative samples for parvovirus. As a result, it was found that the test was performed with good sensitivity even after 24 months, which indicates that the kit was fully dried without an additional absorbent bag.

### EXAMPLE 3

### Test for Distemper using Immunochromatographic Strip and Kit

A nitrocellulose pad and an antibody-gold conjugate pad were produced in the same manner as in Example 1, except that monoclonal antibodies against a distemper virus antigen were used instead of using monoclonal antibodies against canine parvovirus. The absorbent pad was produced as follows. Pulp having a thickness of 1 mm was impregnated with an aqueous solution containing 10 wt % magnesium chloride, and the pulp was left until it was evenly soaked with the absorbent solution. Then, the pulp was dried at 100° C. for 1 hour, and, after attaching a porous membrane (polyethylene) on one side of the pulp, was cut to a size of 20×300 mm, resulting in the production of an immunochromatographic strip and a kit as in Example 1. Then, the immunochromatographic strip and the kit were stored for 1 month, 6 months, 12 months, 18 months, and 24 months, and used for the test of each one of positive and negative samples for distemper virus. As a result, it was found that the test was performed with good sensitivity even after 24 months.

### EXAMPLE 4

### Test for Canine Heartworm using Immunochromatographic Strip and Kit

A nitrocellulose pad and an antibody-gold conjugate pad were produced in the same manner as in Example 1, except that monoclonal antibodies against a canine heartworm antigen were used instead of using monoclonal antibodies against canine parvovirus. The absorbent pad was produced as follows. Pulp having a thickness of 1 mm was impregnated with an aqueous solution containing the same amounts of 10 wt % calcium chloride and 10 wt % magnesium chloride, and the pulp was left until it was evenly soaked with the absorbent solution. Then, the pulp was dried at 100° C. for 1 hour, and, after attaching a porous membrane (polyethylene) on one side of the pulp, was cut to a size of 20×300 mm, resulting in the production of an immunochromatographic strip and a kit as in Example 1. Then, the immunochromatographic strip and the kit were stored for 1 month, 6 months, 12 months, 18 months, and 24 months, and used for the test of each one of positive and negative samples for canine heartworm antigen. As a result, it was found that the test was performed with good sensitivity even after 24 months.

As described, it should be evident that the present invention can be implemented through a variety of configurations in the aforementioned technical field without affecting, influencing or changing the spirit and scope of the present invention. Therefore, it is to be understood that the examples and applications illustrated herein are intended to be in the nature of description rather than of limitation. Furthermore, the meaning, scope and higher conceptual understandings of the present invention as well as modifications and variations that arise therefrom should be understood to be extensions to the present invention.

1. An immunochromatographic strip comprising:
a) an adhesive plastic backing;
b) a sample pad receiving a liquid sample to be analyzed, which is attached on surface of the top portion of the adhesive plastic backing;
c) a conjugate pad coupled to the sample pad, which contains a conjugate specifically binding to an analyte in the liquid sample from the sample pad;
d) a signal detection pad coupled to the conjugate pad, which comprises a signal detection zone to detect the presence of the analyte in the liquid sample and a control zone to confirm whether the liquid sample chromatographically migrates regardless of the presence of the analyte; and
e) an absorbent pad positioned downstream of the signal detection pad, in which the absorbent pad absorbs the liquid sample after the completion of a signal detection reaction,
wherein the absorbent pad comprises a porous backing and an absorbent and the absorbent is dispersed into pores of the porous backing or adsorbed, or coated onto fibers of the porous backing.

2. The immunochromatographic strip according to claim 1, wherein the porous backing is formed of pulp, fiber, polymer, or rubber.

3. The immunochromatographic strip according to claim 1, wherein the absorbent pad further comprises a porous film layer attached on the top portion of the porous backing.

4. The immunochromatographic strip according to claim 1, wherein the signal detection zone is coated with a signal detection material selectively and specifically binding to the products obtained from the binding between the analyte and the conjugates in the conjugate pad, and the control zone is coated with a material specifically binding to the conjugates in the conjugate pad.

5. The immunochromatographic strip according to claim 1, wherein the absorbent is selected from the group consisting of calcium chloride, magnesium chloride, diatomite, bentonite, dolomite, gypsum, silica gel, and a mixture thereof.

6. The immunochromatographic strip according to claim 1, wherein the absorbent is selected from the group consisting of calcium chloride, magnesium chloride, and a mixture thereof.

7. The immunochromatographic strip according to claim 1, wherein the absorbent pad is produced by impregnating the porous backing with an absorbent solution, in which the absorbent is dissolved in water, so that the absorbent is sufficiently soaked in the porous backing, and drying the same, so that the absorbent is adsorbed or coated on the porous backing.

8. An immunochromatographic kit comprising a bottom housing to accommodate an immunochromatographic strip and a top housing complementarily engaged with the bottom housing, wherein the immunochromatographic strip accommodated in the kit is the immunochromatographic strip according to any one of claims 1 to 7, and singly packaged in a packaging material without an additional absorbent bag.

9. The immunochromatographic kit according to claim 8, wherein the top housing comprises a first through-hole formed on the top portion of the sample pad in order to receive a liquid sample; a second through-hole formed on the top portion of the signal detection pad in order to observe the result of the immunoassay in the signal detection pad; and a third through-hole formed on the top portion of the absorbent pad, in which the third through-hole facilitates the absorption of moisture contained in the packaging material by the absorbent in the absorbent pad, prior to using the kit for immunochromatographic assay and facilitates the evaporation of the liquid sample adsorbed by the absorption pad after the immunochromatographic assay.
